(19) United States
(12) Patent Application Publication
(10) Pub. No.: US 2014/0255551 A1
(43) Pub. Date: Sep. 11, 2014

(54) NOVEL 1,3-DIACYLGLCEROL (1,3-DAG) FOR HARD FAT APPLICATIONS

(71) Applicants: Richard John Craven, Orton (CA); Robert Lencki, Guelph (CA)

(72) Inventors: Richard John Craven, Orton (CA); Robert Lencki, Guelph (CA)

(73) Assignee: UNIVERSITY OF GUELPH, GUELPH, ONTARIO (CA)

(21) Appl. No.: 14/236,610

(22) PCT Filed: Aug. 2, 2012
(86) PCT No.: PCT/CA2012/000732

§ 371 Date: May 2, 2014

Related U.S. Application Data
(60) Provisional application No. 61/514,883, filed on Aug. 3, 2011.

(51) Int. Cl. A23G 3/40

(52) U.S. Cl. CPC A23G 3/40
USPC 426/72; 426/607; 426/601

(57) ABSTRACT

The invention provides 1,3-DAGs with melting point ranges of from about 32° C. to about 37° C. for use in a variety of hard fat applications and in particular for the preparation of a cocoa butter substitute for confectionery use. The invention contemplates novel 1,3-DAGs, cocoa butter substitute incorporating such 1,3-DAG compounds, confectionery products and shortenings containing both and methods of making such.

### FIELD OF THE INVENTION

The invention relates to 1,3-DAG for use in a variety of hard fat applications and in particular for the preparation of a cocoa butter substitute for confectionery use. The invention contemplates novel methods for making 1,3-DAG with desirable melt properties as well as cocoa butter substitutes incorporating such 1,3-DAG, confectionery products and shortenings containing both.

### BACKGROUND OF THE INVENTION

All documents, patents, and patent applications referred to herein are incorporated by reference in their entirety.

Due to concerns regarding increasing obesity rates and associated diseases, fat replacement technologies have developed. It is desirable to provide fat replacements as substitutes for some or all dietary fat in food products. One such substitute is a modified dietary fat. A desirable fat replacement is diacylglycerol (DAG). DAG differs markedly from other fat substitute technologies. DAG promotes weight loss and improves blood lipids because DAG (in particular 1,3-DAG) is metabolized differently from triacylglycerols (TAGs) leading to a decrease in the amount of fat being delivered to body stores. In this regard, 1,3-DAG can be referred to as a functional fat that would allow the manufacture of healthier bakery and confectionery products.

The current commercial diacylglycerol (DAG) oil on the market is produced using fatty acids (FA) derived from canola and soybean oils. These oils provide the polyunsaturated (PUFA) and monounsaturated FA (MUFA) that ensure the oil contains no solids, even during extended refrigeration. However, a spread, shortening, confectionery, or confectionery coating must possess a solid fraction that provides the fat product with a distinct melting behavior.

Fat spreads (e.g. tub margarine) have a relatively flat solid fat content (SFC) versus temperature profile so as not to be too hard when removed from the refrigerator, but not too oily at room temperature. Shortening contains a solid fraction that is designed to melt at the appropriate baking temperature, thereby giving pastry containing the shortening a desired flaky texture. In contrast, cocoa butter has a very sharp melting profile so it is hard at room temperature (giving chocolate its desirable snap) but melts completely at body temperature. Too high a melting point would give the cocoa butter an undesirable waxy taste.

In the prior art many fat blends were initially developed based on triacylglycerols. With the growing demand for healthier food products the focus has turned to develop healthier fat blends based on diacylglycerol incorporation as is, for example, disclosed in U.S. Pat. No. 5,879,735, U.S. Pat. No. 5,912,042, U.S. Pat. No. 7,375,240 and U.S. Pat. No. 7,550,615. WO 2010/019598 discloses diacylglycerol semi-solid fats and oils.

One difficulty faced by the prior art is that high yields of 1,3-DAGs cannot be obtained by direct chemical methods because such methods lack positional selectivity in adding the fatty acids at the 1 and 3 positions of glycerol. Multistep reaction sequences involving protection and de-protection are therefore necessary, and large quantities of solvents are used as reaction media and for purification. The classical synthesis method uses 1-monoacylglycerol (1-MAG), which is acylated with an acid chloride in the presence of pyridine and chloroform. Yields of 50-70% are obtained after purification. Improved 1,3-DAG yields (85%) can be obtained by solid phase enzymatic glycerolysis of triglycerides (TAG), but this process is limited to applications where acyl species homogeneity is not important. Furthermore, such methods generally require pure homogeneous synthesized TAG as starting material. 1,3-DAGs can also be synthesized via esterification of glycerol adsorbed beforehand onto a solid support using organic solvents with 1,3-specific lipases. In this case, the synthesis of monoacid 1,3-DAG is done via irreversible acyl transfer using vinyl esters as acyl donors. Similar to the aforementioned chemical method, production of diacid 1,3-DAG using vinyl esters is also a multistep process requiring that the 1(3)-MAG intermediate be isolated prior to the addition of the second fatty acid residue.While DAG fats and oils are known, it is desirable to provide DAGs that more closely mimic the properties of TAG and as such have a melting point that makes the DAG amenable for use as a healthier cocoa butter substitute for various confectionery and food applications while maintaining acceptable mouthfeel. It is also desirable to provide methods of making such DAGs that are simpler than those described above.

### SUMMARY OF THE INVENTION

The invention broadly encompasses novel engineered DAGs, in aspects, a 1,3-DAG that mimics the solid fat content (SFC) versus temperature profiles of common TAG products, thereby providing the same functionality but with enhanced health benefits. The invention also encompasses engineered 1,3-DAGs with desired temperature profiles. The invention further encompasses the use of the 1,3-DAGs in a cocoa butter substitute having widely applicable confectionery and food uses. The invention further encompasses methods of making 1,3-DAGs and cocoa butter substitutes incorporating the same and having desired melting properties. Additionally, the invention encompasses compositions, including confectionery and food products, comprising the engineered 1,3-DAGs and/or cocoa butter substitutes that exhibit desirable sharp melting properties.

According to an aspect of the present invention is an engineered 1,3-DAG having a melting point of up to about 50° C.

According to an aspect of the present invention is novel 1,3-DAG having a sharp melting point range of about 32° C. to 37° C., wherein the 1,3-DAG is essentially pure. In an aspect, is 1,3-palmitoyl-palmitoleoyl-glycerol (16:0-OH-16:1), 1,3-stearoyl-linoleoyl-glycerol (18:0-OH-18:2), or 1,3-palmitoyl-oleoyl-glycerol (16:0-OH-18:1).

According to another aspect of the present invention is novel 1,3-DAG comprising a melting point of up to about 50° C., in aspects up to about 37° C., wherein said 1,3-DAG is essentially pure.

According to another aspect of the present invention is a 1,3-DAG composition comprising 1,3-DAG and one or more of free fatty acids (FFA), monoacylglycerols (MAG),1,2-DAG and TAG. In aspects, the composition has a desirable sharp melt property, said property being in the melting point range of about 32° C. to 37° C. In non-limiting aspects, the FFA<1%; MAG<10%; 1,2-DAG<30% and TAG<20%.

According to an aspect of the present invention is a solid oil/shortening comprising 1,3-DAG as a major component, said composition having melting point range of about 32° C. to about 37° C. The oil/shortening comprises 1,3-DAG having a melting point range of up to about 50° C.

According to another aspect of the present invention is a cocoa butter substitute comprising 1,3-DAG, wherein said cocoa butter substitute has a desirable melt property. In aspects, the cocoa butter substitute has a melting point range of about 32° C. to about 37° C.

According to another aspect of the present invention is a cocoa butter substitute comprising up to about 70% 1,3-DAG and being solid or semi-solid at room temperature. In aspects, said substitute has a melting point of up to about 37° C.

According to further aspects of the present invention are 1,3-palmitoyl-oleoyl-glycerol variants having a melting point of greater than about 37° C.

According to further aspects of the present invention are 1,3-DAG molecules selected from the group consisting of 1,3-myristoyl-palmitoleoyl-glycerol (14:0-OH-16:1); 1,3-myristoyl-oleoyl-glycerol (14:0-OH-18:1); 1,3-palmitoyl-oleoyl-glycerol (16:0-OH-18:1), 1,3-palmitoyl-palmitoleoyl-glycerol (16:0-OH-16:1), 1,3-stearoyl-palmitoleoyl-glycerol (18:0-OH-16:1), 1,3-stearoyl-oleoyl-glycerol (18:0-OH-18:1); 1,3-stearoyl-linoleoyl-glycerol (18:0-Oh-18:2); 1,3-arachidoyl-oleoyl-glycerol (20:0-OH-18:1); and 1,3-arachidoyl-linoleoyl-glycerol (20:0-OH-18:2), wherein the molecules exhibit a melting point of greater than about 37° C.

According to another aspect of the present invention is a cocoa butter substitute comprising an engineered 1,3-DAG molecule having a melting point of greater than about 37° C.

According to another aspect of the present invention is a cocoa butter substitute comprising a blend of one or more 1,3-DAG molecules each of which has a melting point of greater than about 37° C.

In aspects of the invention any one of the following 1,3 DAG molecules can be used as a starting point for engineering to provide a compound with a desired melting profile: 1,3-myristoyl-palmitoleoyl-glycerol (14:0-OH-16:1); 1,3-myristoyl-oleoyl-glycerol (14:0-OH-18:1); 1,3-palmitoyl-palmitoleoyl-glycerol (16:0-OH-16:1), 1,3-palmitoyl-oleoyl-glycerol (16:0-OH-18:1), 1,3-stearoyl-palmitoleoyl-glycerol (18:0-OH-16:1), 1,3-stearoyl-oleoyl-glycerol (18:0-OH-18:1); 1,3-stearoyl-linoleoyl-glycerol (18:0-OH-18:2); 1,3-arachidoyl-oleoyl-glycerol (20:0-OH-18:1); and 1,3-arachidoyl-linoleoyl-glycerol (20:0-OH-18:2).

In aspects of the present invention is an isolated 1,3-palmitoyl-oleoyl-glycerol having a desirable melting point of greater than about 37° C. In aspects, the melting point is about 42° C.

According to another embodiment of the invention is an engineered 1,3-DAG molecule having a melting point of less than about 37° C. In aspects, the compound is 1,3-hexanoyl-palmitoyl-glycerol that melts at about 35° C.

According to another aspect of the invention is a method for making 1,3-DAG with a sharp melting profile.

According to another aspect, there is provided a fat comprising from about 10% to about 90% of one or more 1,3-diacylglycerols (1,3-DAGs), wherein the fat has a melting point of from about 32° C. to about 42° C.

In an aspect, the melting point is from about 32° C. to about 37° C.

In an aspect, the fat is at least about 75% solids at about 20° C. and about 100% liquid at about 37° C.

In an aspect, the one or more 1,3-DAGs are present in the fat in an amount selected from the group consisting of from about 50% to about 90%, from about 60% to about 90%; from about 70% to about 90%; from about 80% to about 90%; and about 90%.

In an aspect, the fat further comprises one or more free fatty acids, monoacylglycerols, 1,2-diacylglycerols, and triacylglycerols.

In an aspect, the free fatty acid content is less than about 1%, the monoacylglycerol content is less than about 10%; the 1,2-diacylglycerol content is less than about 30%, and the triacylglycerol content is less than about 20%.

In an aspect, the one or more 1,3-DAGs have a melting point of up to about 50° C.

In an aspect, the one or more 1,3-DAGs are selected from the group consisting of 1,3-palmitoyl-oleoyl-glycerol, 1,3-butyroyl-palmitoyl-glycerol, 1,3-hexanoyl-palmitoyl-glycerol, 1,3-myristoyl-oleoyl-glycerol, 1,3-stearoyl-oleoyl-glycerol, 1,3-myristoyl-palmitoleoyl-glycerol, 1,3-palmitoyl-palmitoleoyl-glycerol, 1,3-stearoyl-palmitoleoyl-glycerol, 1,3-stearoyl-linoleoyl-glycerol, 1,3-arachidoyl-oleoyl-glycerol, 1,3-arachidoyl-linoleoyl-glycerol, and combinations thereof.

In an aspect, the one or more 1,3-DAGs are in the β polymorphic form, the β polymorphic form, or a combination thereof.

In an aspect, the one or more 1,3-DAGs are in the β polymorphic form.

In an aspect, the one or more 1,3-DAGs are in the β polymorphic form and the fat as a whole is in the β′ polymorphic form.

In an aspect, the one or more 1,3-DAGs are in the β polymorphic form and the fat as a whole is in the 6 polymorphic form.

In an aspect, said fat is a food-grade fat.

In an aspect, said fat is a shortening or spread.

In an aspect, said fat is a cocoa butter substitute.

In an aspect, said fat further comprises one or more additives.

In an aspect, the additives are selected from the group consisting of emulsifiers, flavorants, antioxidants, vitamins, and combinations thereof.

According to another aspect, there is provided a substantially pure 1,3-diacylglycerol (1,3-DAG) composition with a melting point range of from about 21° C. to about 37° C.

In an aspect, the melting point range is from about 32° C. to about 37° C.

In an aspect, the melting point is about 37° C.

In an aspect, the 1,3-DAG is 1,3-palmitoyl-palmitoleoyl-glycerol, 1,3-stearoyl-linoleoyl-glycerol (18:0-OH-18:2), or 1,3-palmitoyl-oleoyl-glycerol (16:0-OH-18:1).

According to another aspect, there is provided a confectionery comprising the fat or the 1,3-DAG described herein.

In an aspect, the confectionery is a chocolate substitute.

According to another aspect, there is provided a method for making the fat described herein, the method comprising:
combining the 1,3-DAG with one or more free fatty acids, monoacylglycerols, 1,2-diacylglycerols, triacylglycerols or combinations thereof, wherein the 1,3-DAG and the one or more free fatty acids, monoacylglycerols, 1,2-diacylglycerols, and triacylglycerols each have a melting point of between about 37° C. to about 50° C.

In an aspect, the one or more free fatty acids, monoacylglycerols, 1,2-diacylglycerols, triacylglycerols, or combinations thereof each have a melting point of between about 37° C. and about 50° C.

In an aspect, the 1,3-DAGs exhibit monotectic or eutectic phase behaviour when combined with the one or more free fatty acids, monoacylglycerols, 1,2-diacylglycerols, triacylglycerols or combinations thereof.

In an aspect, the 1,3-DAGs exhibit eutectic phase behaviour when combined with the one or more free fatty acids, monoacylglycerols, 1,2-diacylglycerols, triacylglycerols or combinations thereof.

According to another aspect, there is provided a cocoa butter substitute comprising at least about 60% of one or more 1,3-diacylglycerols (1,3-DAGs), wherein the cocoa butter substitute has a solid fat content (SFC) versus temperature profile that is substantially the same as that of cocoa butter that does not comprise the one or more 1,3-DAGs.

According to another aspect, there is provided a method for making a 1,3-DAG composition enriched in a 1,3-DAG species, wherein the 1,3-DAG composition has a sharp melting point range of about 32° C. to 37° C., the method comprising:
reacting a monoacylglycerol (M) with a fatty acid (F) to produce a 1,3-DAG composition comprising a mixture of MM, FF, and MF 1,3-DAG species, wherein M and F are selected so that at least one of said 1,3-DAGs will provide the sharp melting point range;
selectively removing from the mixture via crystallization processes all or a portion of two of said 1,3-DAG species to produce the 1,3-DAG composition enriched in a 1,3-DAG species and having the sharp melting point range.

According to another aspect, there is provided a method for making a 1,3-DAG composition having a sharp melting point range of about 32° C. to 37° C., the method comprising:
reacting monolein (O) with palmitic acid (P) to produce a 1,3-DAG composition comprising OO, PP, and OP components;
selectively removing from the mixture via crystallization processes all or a portion of the PP and OO components to produce the 1,3-DAG composition having the sharp melting point range, said 1,3-DAG composition being enriched in the OP component.

The novel 1,3-DAG of the present invention can be made from known raw materials such as MAG and FFA and the reaction catalysts may be, for example, acid or lipase (Craven et al 2011, Journal of the American Oil Chemists Society, DOI:10.1007/s11746-011-1777-0; Craven et al. 2011, Journal of the American Oil Chemists Society, DOI 10.1007/s11746-011-1769-0; Craven et al. 2010, Journal of the American Oil Chemists Society, 87(11), 1281-1291).

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from said detailed description.

### BRIEF DESCRIPTION OF THE DRAWINGS

The present invention will become more fully understood from the detailed description given herein and from the accompanying drawings, which are given by way of illustration only and do not limit the intended scope of the invention.

FIG. 1 is a graph showing how the percent solid fat content (SFC) changes with temperature for typical triacylglycerol-based (TAG-based) commercial fat products.

FIG. 2 is a structural representation of 1,3-palmitoyl-oleoyl-glycerol.

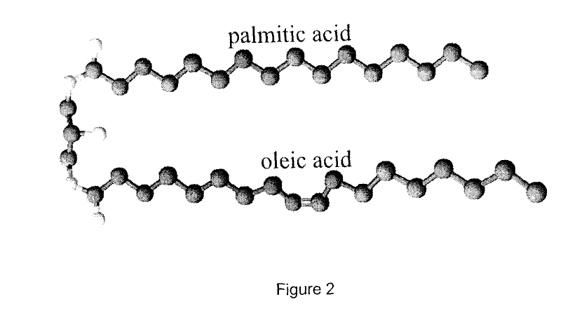

### DETAILED DESCRIPTION OF THE PREFERRED EMBODIMENTS

Provided herein are 1,3-DAGs with desired specific temperature profiles so that they can be used as or in cocoa butter substitutes to be incorporated into foodstuffs such as confectioneries, baked goods and, more specifically, healthier chocolate substitutes. As such, a novel cocoa butter substitute is described that has desirable melt properties and better health properties due incorporation of the 1,3-DAG described herein.

More specifically, the inventors have demonstrated that 1,3-DAGs can be engineered to have a desired melting temperature profile and can thus be used to make a cocoa butter substitute with desired melt properties (i.e. a sharp-melting product). Such 1,3-DAGs and cocoa butter substitutes are healthier than traditionally known triglyceride fat products. Thus the cocoa butter fat described herein, comprising a substantial amount of 1,3-DAGs and having the proper melt properties of a cocoa butter fat without the 1,3-DAGs (e.g., a melting point of about 32 to 37° C.) is desirable.

In an aspect, there is provided a substantially pure 1,3-diacylglycerol (1,3-DAG) composition with a melting point range of from about 21° C. to about 42° C., such as from about 21° C. to about 37° C., such as 27° C. to 37° C., or about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., or about 42° C. In a specific desired aspect, the melting point is about 37° C. An example of such a 1,3-DAG is 1,3-palmitoyl-palmitoleoyl-glycerol, 1,3-stearoyl-linoleoyl-glycerol, or 1,3-palmitoyl-oleoyl-glycerol.

The term “substantially pure” means that there are less than about 15% by weight, such as 14%, 13%, 12%, 11%, 10%, 9%, 8%, 7%, 6%, 5%, 4%, 3%, 2%, or 1%, contaminants in the composition. The 1,3-DAG composition may instead be completely pure. Contaminants include, for example, free fatty acids, MAGs, 1,2-DAGs, and TAGs that may be present in the starting materials or may be introduced during side reactions in the process for preparing the 1,3-DAG composition. It will be understood that the presence of such contaminants will tend to depress the melting point and flatten the SFC versus temperature profile of the 1,3-DAG.

A substantially pure 1,3-DAG composition with a sharp melting point, as described above, could be used directly as a cocoa butter or other hard fat substitute. Any high purity 1,3-DAG with a melting point below about 37° C. is appropriate. 1,3-hexanoyl-palmitoyl-glycerol which melts at approximately 35° C. is one such example. This method of producing a fat substitute does however require extensive purification, and may reduce yield.

A desired aspect of the present invention for producing a sharp-melting fat is therefore to use a 1,3-DAG with a melting point slightly higher than 37° C. (up to about 50° C.) and blend it with other solid fats with similar melting points with which the principal 1,3-DAG demonstrates eutectic phase behavior (mutual melting point depression). This eutectic phase behavior is what occurs with the TAG compounds found in natural cocoa butter, the key fat component in confectionery products such as chocolate bars and coatings.

The main TAGs in cocoa butter are 1-palmitoyl-2-oleoyl-3-stearoyl-glycerol (POS; ˜40%), 1,3-dipalmitoyl-2-oleoyl-glycerol (POP; ˜15%) and 1,3-distearoyl-2-oleoyl-glycerol (SOS; -27%). Individual TAGs all have higher melting points (Tₘ for POS =˜37.5° C., POP=-36.5° C., SOS=43° C.) when measured separately than they do when measured as combined in cocoa butter (Tₘ =˜35° C.) due to this eutectic phase behaviour.

Thus, as has been described above, due to costs and reduced yields that are often associated with producing substantially pure 1,3-DAG compositions, various fats can be combined in order to arrive at a fat blend that exhibits the appropriate melt and physical characteristics due to monotectic or eutectic phase behaviour, typically eutectic phase behaviour.

Accordingly, in an aspect, there is provided a fat comprising one or more 1,3-diacylglycerols (1,3-DAGs), wherein the fat has a melting point of from about 32° C. to about 42° C., such as from about 32° C. to about 37° C., such as about 32° C., about 33° C., about 34° C., about 35° C., about 36° C., about 37° C., about 38° C., about 39° C., about 40° C., about 41° C., or about 42° C. The one or more 1,3-DAGs are present in the composition in amounts up to about 90% by weight, such as from about 10% to 90% and any amount thereinbetween, such as from about 20% to about 70%, from about 25% to about 60%, from about 30% to about 40%, from about 35% to about 50%, from about 50% to about 90%, from about 60% to about 90%; from about 70% to about 90%; from about 80% to about 90%; and about 90%. In an aspect, the one or more 1,3-DAGs are present in the composition in amounts of from about 50% to about 90%.

It is desirable that the fat displays an SFC versus temperature profile that is similar to that of cocoa butter, having a “sharp” melting profile. This means generally that the fat is at least about 75% solids at room temperature (about 20° C.) and about 100% liquid at body temperature (about 37° C.).

The fat described herein may further comprise one or more free fatty acids, monoacylglycerols, 1,2-diacylglycerols, and triacylglycerols. For example, the free fatty acid content may be less than about 1% by weight, the monoacylglycerol content may be less than about 10% by weight; the 1,2-diacylglycerol content may be less than about 30% by weight, and the triacylglycerol content may be less than about 20% by weight. It will be understood that the free fatty acids, monoacylglycerols, 1,2-diacylglycerols, and triacylglycerols will act to reduce the melting point of the 1,3-DAGs through monotectic or eutectic phase behaviour, typically eutectic phase behaviour (mutual melting point depression).

In view of this eutectic phase behaviour, the one or more 1,3-DAGs can be selected to have a higher melting point of up to about 50° C., such as up to about 49° C., up to about 48° C., up to about 47° C., up to about 46° C., up to about 45° C., up to about 44° C., up to about 43° C., up to about 42° C., up to about 41° C., up to about 40° C., up to about 39° C., up to about 38° C., or up to about 37° C. This melting point will be reduced to the desired melting point of from about 32° C. to about 42° C. when other selected components are blended into the fat.

The fat may also comprise a blend of 1,3-DAGs that together contribute to the desired melting point range. For example, a 1,3-DAG comprising palmitic acid and oleic acid moieties (OP) may be combined with a 1,3-DAG comprising only oleic acid moieties (OO) to produce a 1,3-DAG blend having a melting point of about 37° C. In another example, a 1,3-DAG comprising palmitic acid and oleic acid moieties (OP) may be combined with a 1,3-DAG comprising stearic acid and oleic acid moieties (SO) to produce a 1,3-DAG blend having a melting point of about 37° C.

The 1,3-DAGs described herein may independently comprise any fatty acid in the 1 and 3 positions of glycerol, provided the two fatty acids (which may be the same or different) in combination yield a 1,3-DAG with the desired melting point.

Fatty acids that may be used in the 1,3-DAGs described herein include, for example, saturated fatty acids such as palmitic acid, stearic acid, arachidic acid, butyric acid, hexanoic acid, octanoic acid, decanoic acid, lauric acid and myristic acid; monounsaturated fatty acids such as palmitoleic and oleic acid; and polyunsaturated fatty acids such as linoleic acid and linolenic acid. In a desired aspect, the 1,3-DAGs will include one saturated fatty acid, such as palmitic acid, and one monounsaturated fatty acid, such as oleic acid. However, it will be understood that any fatty acid may be used provided it contributes towards reaching the desired melting point range of the 1,3-DAG. Typically, the fatty acids chosen will be suitable for human consumption.

Examples of 1,3-DAGs having melting points within the desired range include 1,3-palmitoyl-oleoyl-glycerol (16:0-OH-18:1; melting point about 43° C.), 1,3-butyroyl-palmitoyl-glycerol (4:0-OH-16:0; melting point about 41° C.), 1,3-hexanoyl-palmitoyl-glycerol (6:0-OH-16:0; melting point about 43° C.), 1,3-myristoyl-oleoyl-glycerol (14:0-OH-18:1; melting point about 41° C.), 1,3-stearoyl-oleoyl-glycerol (18:0-OH-18:1; melting point about 48-49° C.), 1,3-myristoyl-palmitoleoyl-glycerol (14:0-OH-16:1), 1,3-palmitoyl-palmitoleoyl-glycerol (16:0-OH-16:1), 1,3-stearoyl-palmitoleoyl-glycerol (18:0-OH-16:1), 1,3-stearoyl-linoleoyl-glycerol (18:0-OH-18:2), 1,3-arachidoyl-oleoyl-glycerol (20:0-OH-18:1), and 1,3-arachidoyl-linoleoyl-glycerol (20:0-OH-18:2).

A typical 1,3-DAG that is desirably used as a principal component in the fat substitute (e.g, cocoa butter) described herein is 1,3-palmitoyl-oleoyl-glycerol. Pure 1,3-palmitoyl-oleoyl-glycerol melts at approximately 42° C. (Tₑ=42.11; Tₚ=42.38; where Tₑ refers to the extrapolated onset of melting and Tₚ refers to the peak maximum temperature measured by differential scanning calorimetry). This 1,3-DAG can be further combined with other product components having similar melting temperatures (such as fatty acids, MAGs, DAGs, and TAGs, as has been described above), and with which the principal 1,3-DAG displays eutectic phase behaviour. In this way, a fat substitute with an SFC versus temperature profile very similar to natural cocoa butter can be obtained. Natural cocoa butter comprises a variety of saturated fats (e.g. stearic acid, palmitic acid), unsaturated fats such as monounsaturated fats (e.g, oleic acid) and polyunsaturated fats (e.g. linoleic acid).

The 1,3-DAGs may be in any polymorphic form and may include β′ polymorphs, β polymorphs, and combinations thereof. In a specific aspect, the 1,3-DAGs described herein are in the 13 polymorphic form. It will be understood that the 1,3-DAGs may be combined with other components that are in the β′ polymorphic form and, thus the product as a whole will appear to be in the β′ polymorphic form. Therefore, the 1,3-DAGs may themselves be in the 13 polymorphic form but may be a component of a product that is in the β′ polymorphic form. In another aspect, the 1,3-DAGs may be incorporated into a fat that is in the β polymorphic form.

The 1,3-DAGs may be used as racemic mixtures or they may be purified to have specific stereochemistries. In addition, the 1,3-DAGs may be modified in various ways without departing from the scope of the invention.

In an aspect, the fats described herein are food-grade fats that can be used as hard fat substitutes in shortenings, spreads or as cocoa butter substitutes. In a specific aspect, the fats described herein are cocoa butter substitutes. Therefore, included herein are foodstuffs comprising the fats and substantially pure 1,3-DAG compositions described herein. Such foodstuffs may include but are not limited to confectioneries (e.g., chocolates and candies), baked goods (e.g., doughs, cakes, and breads) and the like. In a typical aspect, the fat described herein is a cocoa butter substitute that is used to make a chocolate substitute, as such a substitute would exhibit desired melting properties and would be healthier than chocolate comprising comparable TAGs.

The fats themselves or the foodstuffs made therefrom may comprise additives in order to produce a palatable product. Such additives include, for example, those that would commonly be added to fat products, such as emulsifiers, flavorants, antioxidants, vitamins, and combinations thereof.

As a chocolate substitute comprising the novel fats (cocoa butter) disclosed herein, further additives may be selected from but not limited to chocolate liquor, and cocoa solids. As an unsweetened baking chocolate (bitter chocolate) primarily cocoa solids and the novel cocoa butter described herein would be combined in varying proportions. As a sweet chocolate, cocoa solids, the novel cocoa butter of the invention, other fat, and sugar would be combined. As a sweet chocolate, additionally milk powder or condensed milk could be incorporated. As a white chocolate, the novel cocoa butter of the invention is incorporated, sugar, and milk but no cocoa solids. In any of these embodiments, the novel cocoa butter of the invention is incorporated thus providing health properties and a desirable melt profile as described herein.

The 1,3-DAGs for use herein may be naturally occurring or they may be specifically engineered and designed to have the desired qualities. It will be understood that naturally occurring 1,3-DAGs may not require refining or processing. On the other hand, such naturally occurring 1,3-DAGs may be subject to refining or processing as required and as would be understood.

The 1,3-DAG of the present invention can be made by a variety of production methods as is understood by one of skill in the art using raw materials such as MAG and FAs. Such raw materials can be used to synthesize 1,3-DAG including enzymatic or non-enzymatic means (i.e. using lipases) as is understood by one of skill in the art. Yield and purity is optimized via variation to the experimental conditions, including reaction temperature, pressure and amount of enzyme used.

As is specifically described in Example 4, below, a simple procedure for synthesis of 1,3-DAGs using vinyl esters in solvent has been developed. A monoacylglycerol (e.g. monolein “0”) was mixed with a fatty acid (e.g. palmitic acid “P”) in the presence of a catalyst to produce a 1,3-DAG composition that contains a mixture of all three possible different DAGs (e.g. PP, OO, and OP). Selective crystallization was used to remove the 1,3-DAGs from the composition that would contribute negatively to the melting point (e.g. in Example 4, PP was crystallized out substantially or entirely and a portion of OO was retained in the solvent, as these species would have raised/lowered the melting point beyond the desired range).

More generally, the method involves mixing a monoacylglycerol (designated M herein) with a vinyl ester of a fatty acid (designated F herein). The monoacylglycerol and vinyl ester of a fatty acid are selected so that 1,3-DAGs produced from these moieties will have the desired melt temperature characteristics, either alone or in some combination. For example, the monoacylglycerol could be glycerol monooleate (monolein), glycerol monolinoleate, glycerol monopalmitate, glycerol monostearate, or any other suitable monoacylglycerol. The vinyl ester of a fatty acid could be, for example, a vinyl ester of palmitic acid, stearic acid, myristic acid, or any other suitable fatty acid. It will be understood that a free fatty acid could be used instead or any ester of a fatty acid, such as an ethyl or methyl ester of a fatty acid.

These moieties, M and F, are mixed together in the presence of a suitable enzyme or catalyst that will produce various 1,3-DAGs: MM, MF, and FF. An example of such an enzyme is Lipozyme immobilized enzyme from Mucor miehei. This enzyme may be mobilized or immobilized. The enzyme may be derived from other sources, such as Candida antarctica. The catalyst may be, for example, sodium methoxide.

This step of the method is typically carried out at room temperature (approximately 21° C.) but this temperature may be higher or lower depending upon the reactants and enzyme or catalyst chosen. Similarly, this step is typically conducted at atmospheric pressure but could be carried out at higher or lower pressures, typically lower rather than higher, as desired.

The 1,3-DAGs so produced are mixed in a solvent that is selected to crystallize out one or more of the components, MM, MF, and FF. For example, methyl t-butyl ether at 4° C. was used in Example 4 below in order to crystallize and remove PP from the mixture of PP, OP, and OO. It will be understood that an appropriate solvent and temperature can be selected by a skilled person depending upon which component is desired to be crystallized. For example, the solvent could be an ether (e.g., methyl t-butyl ether or ethyl ether), an alkane (e.g., hexane), a chlorinated solvent (e.g., chloroform) or any combination thereof. This step could be omitted if a component that would be removed by this step is instead desired to be retained in the composition.

Next, the solvent is removed, leaving an oil, solid, or mixture of both containing the remaining 1,3-DAGs. These are mixed with another solvent such as an alcohol (e.g., methanol, ethanol, propanol, etc.), hexane, ethyl acetate, or any combination thereof. Heat may be applied in order to encourage dissolution of the oil in the solvent. After a period of time at a specific temperature, the product will have crystallized and can be removed from the solvent. For example, in Example 4 below, the oil containing OP and OO was mixed with methanol and kept at 4° C. for 5 days in order to crystallize the OP leaving the OO in the solvent.

It will be understood that all of the reaction and storage times and temperatures described above and in specific Example 4 can be modified based upon the disclosure herein and general testing or knowledge in the art in order to arrive at the desired products, yield, and purity levels depending upon the starting materials chosen and the desired end product.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1

### Synthesis of High Purity 1,3-DAG

### 1(3)-monoacylglycerol described for 1(3)-palmitoyl-glycerol

The vinyl ester of palmitic acid (13.4 g) and racemic isopropylideneglycerol (5.0 g) were stirred in 100 mL chloroform containing Lipozyme® immobilized lipase from Rhizomucor miehei (1.0 g) at room temperature (˜22° C.) under nitrogen for three days. Once the reaction was complete, immobilized enzyme was removed by filtration and solvent removed under vacuum. Free 1(3)-palmitoyl-glycerol was produced by gently refluxing in 100 mL 95% ethanol with Amberlyst™15 (wet) resin. The resulting MAG was purified by recrystallization first from acetone and subsequently from hexane/ethyl ether (4:1, v/v). This yielded 7.56 g of product containing 93% 1(3)-palmitoyl-glycerol and 7% 2-palmitoyl-glycerol (by GC).

### 1,3-diacylglycerol described for 1,3-palmitoyl-oleoyl-glycerol

Oleoyl chloride (4.50 g) dissolved in 30 mL methylene chloride was added dropwise to a solution of 1(3)-palmitoyl-glycerol (5.0 g), triethylamine (2.73 g) and N,N-dimethylaminopyridine (0.18 g) in 70 mL methylene chloride stirred in an ice bath. After addition, the solution was stirred at room temperature (˜22° C.) for 3 hours. Solvent was removed under vacuum, the residue taken back up in hexane and filtered, raw product was then crystallized from the filtrate. The product was further purified by flash chromatography with hexane/ethyl acetate (8:2, v/v) to yield 4.14 g of >95% (by GC) pure product.

Final Product 1a

| | | |
|---|---|---|
| Fatty acid profile (%)¹: | 16:0 50% | 18:1 50% |
| Saturates/Unsaturates: | 50/50 | |
| Acylglycerols (%): | DAG: 100% | |
| Tₘ: | 42° C. (tempered) | |

Toxic, noxious and non-food grade reagents and solvents are used in Example 1. Consequently, 1,3-DAG synthesized by this method are not recommended to be consumed as a food product but nonetheless provides an example of a method of high purity production of a 1,3-DAG with a desired melting point for use as a fat substitute.

Example 2 below outlines a process by which two food-grade and readily available raw materials (a MAG and a FFA), can be combined with a commercially-available and food-approved immobilized lipase catalyst to produce the desired target 1,3-DAG. The final product was obtained via crystallization from food-grade hexane.

### Example 2

### Synthesis of a Food Grade Confectionery Fat using Immobilized Lipase

Technical grade (˜90%) oleic acid (25 g) was combined with 25 g Dimodan® HP-K-A (distilled monoglyceride from hydrogenated palm oil; with approximately 60% palmitic and 40% stearic acids) and Lipozyme® immobilized lipase from Rhizomucor miehei (2.5 g) at approximately 50° C. for 3 hours. Dry nitrogen was bubbled through the reaction vessel under reduced pressure to provide agitation and remove resultant water. Crude product was dissolved in a minimum of hexane and catalyst was removed by filtration. Fractions were obtained by crystallization from hexane at ˜22° C., 4° C. and -20° C.

Final Product 2a

| | | | | |
|---|---|---|---|---|
| lab book | JC3-08c | | | |
| reference: | | | | |
| Fatty add | 16:0 29% | 18:0 23% | 18:1 47% | 18:2 1% |
| profile (%)¹: | | | | |
| Saturates/ | 52/48 | | | |
| Unsaturates: | | | | |
| Acylglycerols | SM²: 9.72% | | DAG: 86.57% | TAG: 3.33% |
| (%): | | | | |
| Tₘ: | 33° C. | | | |
| | (tempered) | | | |

Final Product 2b

| | | | | |
|---|---|---|---|---|
| lab book | JC3-11b | | | |
| reference: | | | | |
| Fatty acid profile | 16:0 34% | 18:0 24% | 18:1 41% | 18:2 1% |
| (%)¹: | | | | |
| Saturates/ | 58/42 | | | |
| Unsaturates: | | | | |
| Acylglycerols | SM²: 8.64% | | DAG: 91.36% | TAG: 0% |
| (%): | | | | |
| Tₘ: | 34° C. | | | |
| | (tempered) | | | |

Notes:
¹16:0 ≡ palmitic acid, 18:0 ≡ stearic acid, 18:1 ≡ oleic acid, 18:2 ≡ linoleic acid
²SM ≡ starting material i.e. fatty acid and MAG

### Shortenings and Consumer Spreads

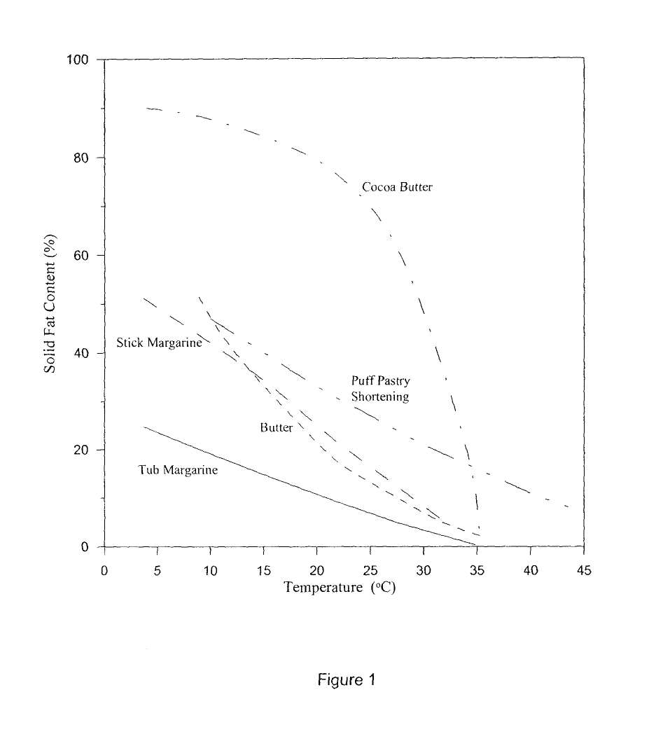

Shortenings and consumer spreads generally have a much flatter SFC versus temperature profile than confectionery fats (FIG. 1). As a result, they require a more complex mixture of TAG with a much broader spectrum of melting points. For example, butterfat contains >300 TAG species whereas a typical shortening contains <100 chemical species. The compositions of commercial shortenings vary widely depending the manufacturer and application. While lipase could be used to produce a 1,3-DAG fat shortening or spread, production using a cheaper acid catalyst is also feasible (see Example 3).

### Example 3

### Synthesis of a Food Grade Shortening using an Acid Catalyst

Technical grade (˜90%) oleic acid (23.3 g) was combined with 28.3 g Dimodan® HP-K-A (distilled monoglyceride from hydrogenated palm oil; with approximately 60% palmitic and 40% stearic acids) and Amberlyst™ 15 (wet) catalyst (5 g) at approximately 80° C. for 6 hours. Dry nitrogen was bubbled through the reaction vessel under reduced pressure to provide agitation and remove resultant water. Crude product was dissolved in a minimum of hexane and catalyst was removed by filtration. Fractions obtained by crystallization from hexane at ˜22° C., 4° C. and -20° C.

Final Product 3a

| | | | | |
|---|---|---|---|---|
| lab book reference: | JC3-11a | | | |
| Fatty acid profile | 14:0 1% | 16:0 52% | 18:0 42% | 18:1 5% |
| (%): | | | | |
| Saturates/ | 95/5 | | | |
| Unsaturates: | | | | |
| Acylglycerols (%): | SM: 19.6% | | DAG: 80.4% | TAG: 0% |
| Tₘ: | 64° C. | | | |
| | (tempered) | | | |

### Example 4

### Synthesis of 1,3-DAGs using vinyl esters in Solvent

A solution of glycerol monooleate (14.5 g) and vinyl ester of palmitic acid (16 mL) in 250 mL methyl t-butyl ether containing Lipozyme immobilized enzyme from Mucor miehei (0.5 g) was gently stirred at room temperature (approximately 21° C.) and atmospheric pressure. After 6 hours the enzyme was filtered from the solution and the filtrate was stored at 4° C. After approximately two weeks, 5.72 g of white crystals were filtered from the solution. Solvent was removed from the filtrate using a rotary evaporator. The resulting light oil was dissolved in 750 mL methanol by applying heat. After 5 days at 4° C., the product (10.9 g of JC3-44b) was filtered from the alcohol solution.

Final Product 44b

| | | |
|---|---|---|
| Lab book reference: | JC3-44b | |
| Fatty acid profile (%): | 16:0 54% | 18:1 46% |
| Saturates/Unsaturates: | 54/46 | |
| Acylglycerols (%): | SM: 2% | DAG: 98% (10% |
| | | 16:0-OH-16:0 and |
| | | 88% 16:0-OH-18:1) |
| Tₘ: | Tₒₙₛₑₜ = 36.52° C. | Tₚₑₐₖ = 40.02° C. |

1.-36. (canceled)

37. A fat comprising from about 10% to about 90% of one or more 1,3-diacylglycerols (1,3-DAGs), wherein the fat has a melting point of from about 32° C. to about 42° C.

38. The fat of claim 37, wherein the melting point is from about 32° C. to about 37° C.

39. The fat of claim 37, wherein the fat is at least about 75% solids at about 20° C. and about 100% liquid at about 37° C.

40. The fat of claim 37, wherein the one or more 1,3-DAGs are present in the fat in an amount selected from the group consisting of from about 50% to about 90%, from about 60% to about 90%, from about 70% to about 90%, from about 80% to about 90% and about 90%.

41. The fat of claim 37, further comprising one or more free fatty acids, monoacylglycerols, 1,2-diacylglycerols and triacylglycerols.

42. The fat of claim 41, wherein the free fatty acid content is less than about 1%, the monoacylglycerol content is less than about 10%; the 1,2-diacylglycerol content is less than about 30%, and the triacylglycerol content is less than about 20%.

43. The fat of claim 37, wherein the one or more 1,3-DAGs have a melting point of up to about 50° C.

44. The fat of claim 43, wherein the one or more 1,3-DAGs are selected from the group consisting of 1,3-palmitoyl-oleoyl-glycerol, 1,3-butyroyl-palmitoyl-glycerol, 1,3-hexanoyl-palmitoyl-glycerol, 1,3-myristoyl-oleoyl-glycerol, 1,3-stearoyl-oleoyl-glycerol, 1,3-myristoyl-palmitoleoyl-glycerol, 1,3-palmitoyl-palmitoleoyl-glycerol, 1,3-stearoyl-palmitoleoyl-glycerol, 1,3-stearoyl-linoleoyl-glycerol, 1,3-arachidoyl-oleoyl-glycerol, 1,3-arachidoyl-linoleoyl-glycerol, and combinations thereof.

45. The fat of claim 44, wherein the one or more 1,3-DAGs are in the β′ polymorphic form, the β polymorphic form, or a combination thereof.

46. The fat of claim 1, wherein the 1,3-DAGs exhibit monotectic or eutectic phase behaviour when combined with the one or more free fatty acids, monoacylglycerols, 1,2-diacylglycerols, triacylglycerols or combinations thereof.

47. The fat of claim 37, wherein said fat is a food-grade fat selected from the group consisting of a shortening, a spread, and a cocoa butter substitute.

48. The fat of claim 47, further comprising one or more additives, selected from the group consisting of emulsifiers, flavorants, antioxidants, vitamins and combinations thereof.

49. A confectionery comprising the fat of claim 37.

50. The confectionery of claim 49, wherein the confectionery is a chocolate substitute and optionally comprises one or more additives selected from the group consisting of emulsifiers, flavorants, antioxidants, vitamins and combinations thereof.

51. A composition consisting essentially of 1,3-diacylglycerol (1,3-DAG), wherein the composition has a melting point range of from about 21° C. to about 37° C.

52. The composition of claim 51, wherein the 1,3-DAG is selected from the group consisting of 1,3-palmitoyl-palmitoleoyl-glycerol, 1,3-stearoyl-linoleoyl-glycerol, 1,3-palmitoyl-oleoyl-glycerol and combinations thereof.

53. A cocoa butter substitute comprising at least about 60% of one or more 1,3-diacylglycerols (1,3-DAGs), each of which has a melting point of greater than 37° C. , wherein the cocoa butter substitute has a solid fat content (SFC) versus temperature profile that is substantially the same as that of cocoa butter that does not comprise the one or more 1,3-DAGs.

54. The cocoa butter substitute of claim 53, wherein the 1,3-DAGs are naturally occurring or are engineered.

55. A method for making a fat according to claim 38, the method comprising:
reacting a monoacylglycerol (M) with a fatty acid (F) to produce a 1,3-DAG mixture comprising MM, FF, and MF 1,3-DAG species, wherein M and F are selected so that at least one of said 1,3-DAG species will provide the sharp melting point range;
selectively removing from the mixture via crystallization processes all or a portion of two of said 1,3-DAG species to produce the fat.

56. A method for making a fat according to claim 38, the method comprising:
reacting monolein (0) with palmitic acid (P) to produce a 1,3-DAG composition comprising OO, PP, and OP components;
selectively removing from the mixture via crystallization processes all or a portion of the PP and OO components to produce the fat, said fat being enriched in the OP component.

57. A confectionery comprising the cocoa butter substitute of claim 53.
